(19) United States
(12) Patent Application Publication
(10) Pub. No.: US 2016/0296382 A1
(43) Pub. Date: Oct. 13, 2016

(54) Methods and Devices for Controlling Hemorrhage During Minimally Invasive Surgical Procedures

(71) Applicant: Jeff Spillane, Yarmouthport, MA (US)

(72) Inventor: Jeff Spillane, Yarmouthport, MA (US)

(21) Appl. No.: 15/093,647

(22) Filed: Apr. 7, 2016

Related U.S. Application Data
(60) Provisional application No. 62/143,861, filed on Apr. 7, 2015.

(51) Int. Cl. A61F 13/36; A61B 17/00

(52) U.S. Cl. CPC A61F 13/36; A61B 17/0057; A61B 2017/00637

(57) ABSTRACT

In various aspects, methods and systems in accordance with the present teachings are provided that enable the tamponade of an internal hemorrhage site that can potentially occur during a closed, minimally invasive surgical procedure. In the unfortunate instance that an acute hemorrhage requires unscheduled conversion to an open surgery, the exemplary methods and systems described herein can enable the surgeon to prevent excessive blood loss from the hemorrhaged vessel during the conversion or repair process. Whereas in open surgeries hemorrhage is typically provided by a gauze sponge held by a set of ringed clamps (e.g., a sponge stick), the small diameter access ports through trocars used in minimally invasive surgeries can make it difficult to deliver sufficient gauze or other absorptive material to properly, temporarily occlude the hemorrhaged vessel. Moreover, because placing a clamp around the bleeding vessel (e.g., with the laparoscopic/robotic surgical tools present at the surgical site) can be dangerous due to poor visibility and the lack of isolation of the vessel, devices in accordance with the present teachings can be placed in compression with the hemorrhage site to slow blood loss during the conversion to an open surgery.

### RELATED APPLICATIONS

This application claims priority to U.S. Provisional Application Ser. No. 62/143,861 filed on Apr. 7, 2015, the content of which are hereby incorporated by reference in its entirety.

### FIELD

The teachings herein relate to methods and devices for use in minimally invasive surgical procedures, and more particularly to methods and devices for controlling hemorrhage (e.g., due to rupture of a blood vessel) during minimally invasive surgical procedures.

### BACKGROUND

Minimally invasive surgical procedures such as laparoscopic surgery, endo-assisted procedures, video-assisted thorascopic surgery (VATS), and other similar procedures generally utilize fiber optic cameras and laparoscopic-specific instrumentation delivered to a surgical site (e.g., the abdominal cavity, thoracic cavity, etc.) through cannulas inserted into small incisions in the skin. Over the last several decades, these minimally invasive surgical techniques have become increasingly common due to potential benefits over traditional open techniques, including reduced pain, shorter post-operative recovery time (and decreased duration of hospital stays), and minimal scarring. Recently-introduced robotic systems such as the da Vinci™ Surgical System (marketed by Intuitive Surgical, Inc. of Sunnyvale, Calif., U.S.A.) can further provide improved 3D vision of the operative field and additional degrees of freedom that allow for more complex movements of the surgical instruments in the limited space afforded during minimally invasive surgical procedures.

Despite major advancements in both minimally invasive surgical procedures and surgeons' skill in utilizing robotic systems for the same, it is inevitable that some minimally invasive surgical procedures must be intra-operatively converted to open surgeries due to surgical complications, technical problems, or oncological conditions. One of the more dreaded instances of intra-operative complications requiring conversion to open surgery (of particular concern during the operation of complex robotic surgical systems) involves intra-operative hemorrhage that can lead to the pooling of blood at the surgical site, thereby occluding the surgeon's field of view so as to make corrective action difficult. With traditional open surgeries, such complications are immediately addressed with the application of direct pressure to the vessel (“tamponade”) by a gauze sponge held by a set of ringed clamps (e.g., a sponge stick), while a plan is made to block, occlude, ligate, or otherwise repair the damaged vessel. However, in light of the increasingly smaller access ports that are being utilized in minimally invasive surgeries, it can be difficult to deliver sufficient gauze or other absorptive material through the cannula for tamponade of the hemorrhage site. Because placing a clamp around the bleeding vessel (e.g., with the laparoscopic/robotic surgical tools present at the surgical site) can also be dangerous due to poor visibility and the lack of isolation of the vessel, conversion to an open surgery may therefore be necessary to address the hemorrhage. Conversion to an open surgery can take up to about 10 minutes between the decision to convert and when open surgery resumes (time is required to retrieve open surgery equipment, remove the robotic arms, prepare the patient and the room, and make an incision in the patient to access the surgical site), while continued bleeding from the vascular injury can possibly cause life-threatening blood loss. Though robotic arms may be used to slow the bleeding, the continued presence of the robot in the limited space of the operating room environment can also impede/interfere with the conversion process.

Accordingly, there remains a critical need for controlling severe hemorrhage during minimally invasive surgical procedures, for example, during conversion to an open procedure.

### SUMMARY

In various aspects, methods and systems in accordance with the present teachings are provided enabling tamponade of an internal hemorrhage site that can occur during a closed, minimally invasive surgical procedure so as to stabilize the hemorrhage site. Because such an acute hemorrhage may need to be addressed by converting to an open surgery, methods and systems in accordance with various aspects of the present teachings can prevent excessive blood loss from the hemorrhaged vessel, while providing the surgeon sufficient time to convert from an endoscopic surgery to an open surgery and, for example, to remove robotic arms. In accordance with various aspects of the present teachings, methods and devices for applying tamponade to an internal hemorrhage site during a laparoscopic procedure are provided in which an absorbent pad coupled to the distal end of an elongate shaft can be delivered through a trocar. During delivery (e.g., through the trocar), the absorbent pad can be disposed in a delivery configuration in which it extends substantially along the longitudinal axis of the elongate shaft. After delivering the absorbent pad to the internal surgical site, the absorbent pad can be actuated into a deployed configuration effective to apply tamponade to the hemorrhage site. In some aspects, for example, the actuation can move the distal end of the absorbent pad toward the proximal end of the elongate shaft (e.g., by bending or folding the absorbent pad) such that the absorbent pad in the deployed configuration has a wider and/or thicker distal portion relative to the distal end of the absorbent pad when in the delivery configuration. In various aspects, this distal portion can then be applied to the hemorrhage site (e.g., compressed against the hemorrhage site) to reduce excess blood loss during conversion or repair.

In accordance with various aspects of the present teachings, a device for applying tamponade to an internal hemorrhage site during a laparoscopic procedure is provided, the device comprising an elongate shaft extending along a longitudinal axis from a proximal end to a distal end and an absorbent pad disposed at or coupled to the distal end of the elongate shaft, the absorbent pad extending in a delivery configuration along the longitudinal axis from a proximal end to a distal end. The device can also include an actuation mechanism extending from the proximal end of the elongate shaft to the absorbent pad, wherein actuation of the actuation mechanism is configured to move the absorbent pad between the delivery configuration and a deployed configuration for applying tamponade to the hemorrhage site. For example, in the deployed configuration, the absorbent pad can be folded such that its distal and proximal ends can be disposed adjacent one another.

The absorbent pad can have a variety of configurations. By way of example, in some aspects, the absorbent pad can exhibit a first length along the central longitudinal axis in the delivery configuration and a second length along the central longitudinal axis in the deployed configuration, the first length being greater than the second length. In some related aspects, the first length is about twice the second length. Additionally or alternatively, a distal portion of the absorbent pad can exhibit an increased thickness in the deployed configuration relative to the proximal end of the absorbent pad in the delivery configuration.

The actuation mechanism can also have a variety of configurations and can be coupled to the absorbent pad in a variety of manners. In some aspects, for example, the actuation mechanism can be configured to fold the absorbent pad. By way of non-limiting example, the actuation mechanism can be configured to rotate a distal portion of the absorbent pad about the distal end of the elongate shaft. Additionally in some aspects, the distal portion of the elongate shaft can have a reduced diameter relative to a proximal portion of the elongate shaft, wherein the reduced-diameter distal portion of the elongate shaft is disposed within the absorbent pad. In various aspects, the distal portion of the elongate shaft can be more flexible relative to the proximal portion of the elongate shaft (e.g., such that actuation of the actuation mechanism can bend or curve the distal portion of the elongate shaft).

In accordance with various aspects of the present teachings, the actuation mechanism can comprise a pull lever extending from the absorbent pad to the proximal end of the elongate shaft (e.g., which can be actuated by a user at the proximal end of the elongate shaft). By way of example, the pull lever can be pulled (e.g., via a handle) so as to move the absorbent pad from the delivery configuration to the deployed configuration. After deployment, the absorbent pad can then be compressed against the hemorrhage site to occlude bleeding. In some aspects, the pull lever can be sufficiently rigid to aid delivery of the absorbent pad in the delivery configuration through a trocar extending through a patient's skin surface and/or to enable the distal portion of the absorbent pad to be applied with pressure to a hemorrhage site in the deployed configuration. By way of example, after actuating the pull lever, a distal portion of the absorbent pad in the deployed configuration can have an increased surface area relative to a distal portion of the absorbent pad in the delivery configuration.

In some aspects, a distal portion of the actuation mechanism can extend through the absorbent pad and be coupled to the distal end of the elongate shaft. In such configurations, the distal portion of the actuation mechanism can be sufficiently rigid to aid delivery of the absorbent pad through the trocar extending through a patient's skin surface. In some related aspects, the distal portion of the actuation mechanism extending through the absorbent pad can comprise at least two segments having different rigidities. For example, a less rigid portion can effectively function as a hinge within the absorbent pad upon actuation of the actuation mechanism. In some aspects, the actuation mechanism can extend through the distal end of the elongate shaft, along an external surface of the absorbent pad, into the distal end of the absorbent pad, through the absorbent pad, out of the proximal end of the absorbent pad, and can be coupled to the distal end of the elongate shaft.

In some aspects of the present teachings, the elongate shaft can define one or more lumen extending therethrough. By way of example, the elongate shaft can comprise a sidewall disposed about a lumen through which the longitudinal axis extends, wherein the sidewall defines an opening proximal to the distal end of the elongate shaft. In various aspects, the actuation mechanism can extend through this opening of the sidewall and couple to at least one of the distal end of the absorbent pad and the distal end of the elongate shaft. By way of non-limiting example, in some aspects the absorbent pad can comprise a distal cap to which the actuation mechanism is coupled.

In accordance with various aspects of the present teachings, the actuation mechanism can be configured to pull the distal end of the absorbent pad toward the proximal end of the absorbent pad substantially along the longitudinal axis. By way of example, the actuation mechanism can extend through the absorbent pad and can be coupled to a distal end thereof. In some aspects, the absorbent pad can comprise a distal cap to which the actuation mechanism is coupled.

The absorbent pad can have a variety of configurations and can be made of a variety of materials. The absorbent pad can be gauze or a sponge formed in the shape of a cylinder or gauze rolled into a cylinder to ease passage through the trocar.

Devices in accordance with the present teachings can additionally include one or more additional features to aid in the treatment of an internal hemorrhage. By way of non-limiting example, one or more lumen can extend through the elongate shaft and can be in fluid communication with the absorbent pad. In various aspects, the lumen can be coupled to a negative pressure source for application of suction through the absorbent pad and/or to a fluid source for application of hemostatic agents to the hemorrhage site through the absorbent pad.

In accordance with various aspects of the present teachings, methods of applying tamponade to an internal hemorrhage site during a laparoscopic procedure are provided that comprise delivering an absorbent pad (e.g., gauze, sponge) in a first configuration through a trocar extending through a skin surface to an internal hemorrhage site, the absorbent pad being coupled to a distal end of an elongate shaft extending along a longitudinal axis from a proximal end to the distal end, wherein the absorbent pad extends substantially along the longitudinal axis from a proximal end to a distal end in the first configuration. The method can also comprise deploying the absorbent pad by moving the absorbent pad from the first configuration to a second configuration in which the distal end of the absorbent pad is disposed adjacent to the proximal end of the absorbent pad. Thereafter, pressure can be applied to the internal hemorrhage site with the absorbent pad in the second configuration. In various aspects, the absorbent pad exhibits a first length along the central longitudinal axis in the first configuration and a second length along the central longitudinal axis in the second configuration, the first length being greater than the second length. For example, the first length can be about twice the second length. Additionally, a distal portion of the absorbent pad can have an increased thickness in the second configuration relative to the proximal end of the absorbent pad in the first configuration. In various aspects, methods in accordance with the present teachings can further comprise converting the laparoscopic procedure to an open procedure while maintaining pressure to the internal hemorrhage site with the absorbent pad in the second configuration. In some aspects, the laparoscopic procedure can be a robot-assisted laparoscopic procedure, wherein the method further comprises disengaging the robot from the patient prior to making a conversion incision.

In some aspects, deploying the absorbent pad comprises pulling a pull lever extending from the distal end of the absorbent pad to the proximal end of the elongate shaft. Additionally or alternatively, deploying the absorbent pad comprises folding the absorbent pad. By way of example, a distal portion of the absorbent pad can be rotated about the distal end of the elongate shaft. In related aspects, the distal portion of the elongate shaft can have a reduced diameter relative to a proximal portion of the elongate shaft, and the reduced-diameter distal portion of the elongate shaft can extend at least partially through the absorbent pad. In some related aspects, the distal portion of the elongate shaft can be more flexible relative to the proximal portion of the elongate shaft such that the distal portion bends while deploying the absorbent pad. Additionally or alternatively, in some aspects, the elongate shaft comprises a sidewall disposed about the longitudinal axis, the sidewall defining an opening proximal to the distal end of the elongate shaft, wherein deploying the absorbent pad comprises pulling a pull lever coupled to the distal end of the absorbent pad through the opening. In some aspects, the method can further comprise locking the pull lever to maintain the absorbent pad in the second configuration.

In various aspects of the present teachings, deploying the absorbent pad can comprise pulling the distal end of the absorbent pad toward the proximal end of the absorbent pad substantially along the longitudinal axis.

In some aspects, the methods described herein can additionally comprise coupling a lumen extending through the elongate shaft and in fluid communication with the absorbent pad to a negative pressure source so as to apply suction through the absorbent pad. Additionally or alternatively, a lumen extending through the elongate shaft can be coupled to a fluid source, for example, to deliver a hemostatic agents to the hemorrhage site through the absorbent pad.

These and other features of the applicant's teaching are set forth herein.

### BRIEF DESCRIPTION OF THE DRAWINGS

The foregoing and other objects and advantages of the invention will be appreciated more fully from the following further description, with reference to the accompanying drawings. The skilled person in the art will understand that the drawings, described below, are for illustration purposes only. The drawings are not intended to limit the scope of the applicant's teachings in any way.

FIG. 1, in schematic diagram, illustrates an exemplary device for applying tamponade to an internal hemorrhage site in a delivery configuration in accordance with various aspects of the applicant's teachings.

FIG. 2, in schematic diagram, illustrates the device of FIG. 1 in a deployed configuration.

FIGS. 3A, 3B, and 3C schematically depict the delivery, deployment, and tamponade of an internal hemorrhage site through a trocar, respectively, using the device of FIG. 1 in accordance with various aspects of the present teachings.

FIG. 4 depicts another exemplary device for applying tamponade to an internal hemorrhage site in accordance with various aspects of the applicant's teachings.

FIG. 5 depicts another exemplary device for applying tamponade to an internal hemorrhage site in accordance with various aspects of the applicant's teachings.

FIGS. 6A and 6B schematically depict another exemplary device for applying tamponade to an internal hemorrhage site in accordance with various aspects of the present teachings.

FIGS. 7A-E schematically depict another exemplary device for applying tamponade to an internal hemorrhage site in accordance with various aspects of the present teachings.

FIGS. 8A-H schematically depict another device for applying tamponade to an internal hemorrhage site in accordance with various aspects of the present teachings.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner.

In various aspects, methods and systems in accordance with the present teachings are provided that enable the tamponade of an internal hemorrhage site that can potentially occur during a closed, minimally invasive surgical procedure. In the unfortunate instance that an acute hemorrhage requires unscheduled conversion to an open surgery, the exemplary methods and systems described herein can enable the surgeon to prevent excessive blood loss from the hemorrhaged vessel during the conversion or repair process. Whereas in open surgeries hemorrhage is typically provided by a gauze sponge held by a set of ringed clamps (e.g., a sponge stick), the small diameter access ports through the trocars typically used in minimally invasive surgeries can make it difficult to deliver sufficient gauze or other absorptive material to properly, temporarily occlude the hemorrhaged vessel. Moreover, because placing a clamp around the bleeding vessel (e.g., with the laparoscopic/robotic surgical tools present at the surgical site) can be dangerous due to poor visibility and the lack of isolation of the vessel, devices in accordance with the present teachings can be placed in compression with the hemorrhage site to slow blood loss during the conversion to an open surgery.

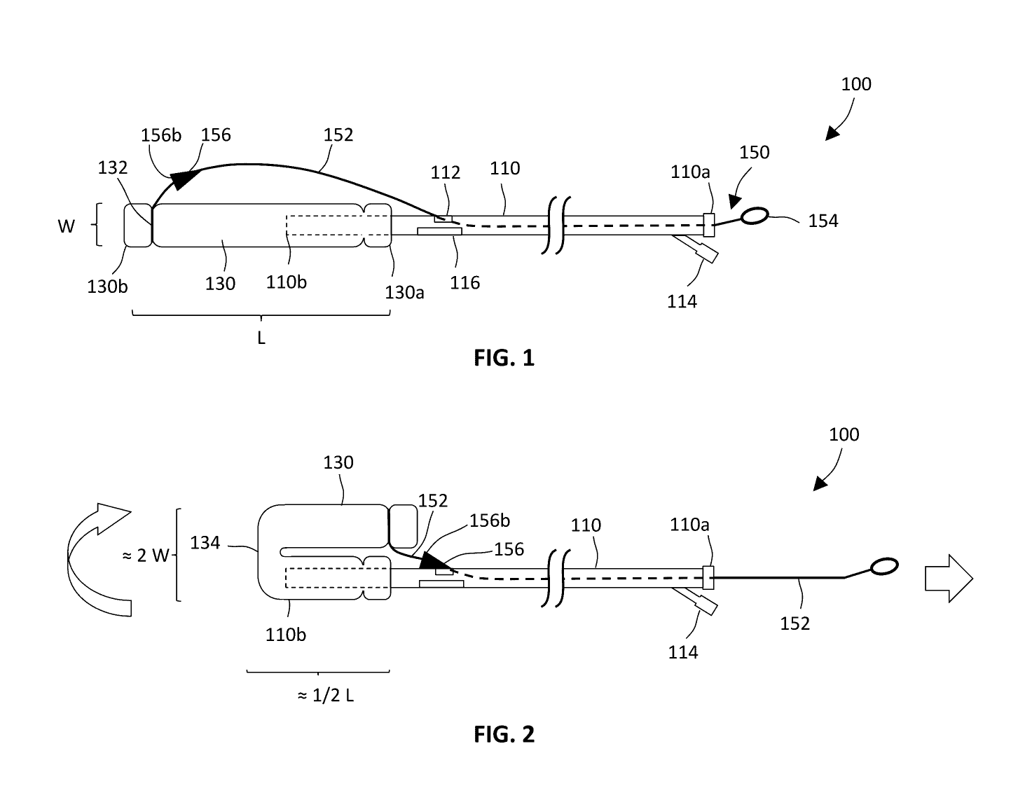

In accordance with various aspects of the present teachings, methods and devices for applying tamponade to an internal hemorrhage site during a laparoscopic procedure are provided in which an absorbent pad coupled to the distal end of an elongate shaft can be delivered through a trocar in a first configuration Upon being ejected from the trocar (e.g., to a location adjacent the surgical site), the absorbent pad can be reconfigured into a second configuration such that the absorbent pad has a distal portion exhibiting sufficient surface area of absorbent material for applying direct pressure to the hemorrhage site (e.g., compression against the hemorrhage site). With reference now to FIGS. 1 and 2, an exemplary embodiment of a device for applying tamponade to an internal hemorrhage site is depicted. As shown in the figures, the exemplary device 100 generally comprises an elongate shaft 110, an absorbent pad 130, and an actuation mechanism 150 configured to move the absorbent pad 130 between a delivery configuration as shown in FIG. 1 and a deployed configuration as shown in FIG. 2.

It will be appreciated by a person skilled in the art that the elongate shaft 110 can have a variety of lengths but is generally configured to extend along a central longitudinal axis from a proximal end 110a that can be disposed external to a patient (e.g., for gripping by the operator or coupled to a handle) to a distal end 110b that can be disposed within the patient. In accordance with various aspects of the present teachings, the elongate shaft can have a length in a range of about 12 inches to about 22 inches, though longer or shorter shafts can also be suitable depending for example on the depth of the surgical site to which the absorbent pad 130 is to be delivered. Likewise, though the exemplary shaft 110 is depicted as having a circular cross-sectional shape, it will be appreciated that the elongate shaft 110 can have a variety of cross-sectional shapes (e.g., round, square, oval) and sizes. In various aspects, however, it may be preferable that the elongate shaft 110 is circular in cross-section so as to ease insertion through a trocar having a circular seal and/or cannula. Similarly, the elongate shaft 110 can have a variety of cross-sectional dimensions but is generally configured to be able to extend through the cannula to the surgical site (e.g., the shaft 110 has a smaller diameter than the cannula through which the shaft 110 is inserted). In accordance with various aspects of the present teachings, the exemplary elongate shaft can have a substantially constant cross-sectional diameter (e.g., less than or equal to 15 mm, about 15 mm, about 12 mm, about 11 mm, in a range of about 5 mm to about 8 mm).

In accordance with various aspects of the present teachings, the elongate shaft 110 can comprise one or more lumens that extend at least partially along the length of the elongate shaft 110 (e.g., a hollow cylindrical body). By way example, as shown in FIG. 1, the elongate shaft can include a first lumen through which the actuation mechanism 150 can extend from the proximal end 110a of the elongate shaft 110 to a distal opening 112 in the sidewall of the elongate shaft 110, as discussed in detail below. Further, the elongate shaft 110 can additionally include one or more additional lumens that extend through the distal end 110b of the elongate shaft so as to be in fluid communication with the absorbent pad 130. It will be appreciated by a person skilled in the art in light of the present teachings that these one or more additional lumens can be coupled (e.g., via port 114) to a fluid supply (not shown) to enable a fluid (e.g., a solution containing a hemostatic agent) to be delivered to the absorbent pad (e.g., to be exuded therefrom at the hemorrhage site) and/or a negative pressure source (not shown) such that suction can be applied to the hemorrhage site.

As shown in FIGS. 1 and 2, the elongate shaft 110 can be formed from a variety of materials that are of sufficient rigidity and strength to push the absorbent pad 130 through and eject the absorbent pad 130 from the distal end of a trocar as otherwise discussed herein. It will further be appreciated that the shaft 110 can comprise a plurality of sections formed from the same or different materials as one another so as to provide the elongate shaft 110 with different rigidities along its length. In accordance with one aspect of the present teachings, the elongate shaft 110 can comprise a hollow carbon fiber or steel shaft having an external diameter less than about 15 mm and an internal diameter in a range of from about 5 mm to about 8 mm. In accordance with various aspects of the present teachings, the sidewall of the elongate shaft can also be reinforced with additional materials (the same or different) from the remainder of the material of the elongate shaft 110 or different, stronger materials so as to provide for increased strength at various stress points of the elongate shaft. As noted above, for example, the actuation mechanism can extend through an opening 112 in the sidewall of the elongate shaft 110. To avoid failure of the shaft 110 at this opening 112, the shaft can be reinforced in proximity to the opening 112 with an additional layer of steel, for example.

As shown in FIGS. 1 and 2, the device 100 also generally comprises an absorbent pad 130 that is disposed at and/or coupled to and extends distally from the distal end 110b of the elongate shaft 110. The absorbent pad 130 can comprise a variety of absorbent materials, which are typically compliant, compressible, and/or flexible in accordance with the present teachings such that the absorbent pad 130 can be compressed against the damaged vessel without causing damage thereto, while nonetheless being effective to stop and/or slow the hemorrhage. Exemplary materials suitable for use in accordance with the present teachings include without limitation compressible porous materials, compressed cellulose sponge, natural sponge, synthetic sponge, polyurethane foam, collagen, and compacted gauze or other natural fibrous materials (e.g., cotton). In accordance with various aspects of the present teachings, the absorbent pad 130 can contain one or more radio-opaque markers so as to prevent any gauze from being left behind following the surgery.

The absorbent pad 130 can have a variety of shapes and sizes, but is generally limited by the dimensions of the trocar through which the absorbent pad 130 is delivered to the surgical site. By way of non-limiting example, the absorbent pad 130 can be cylindrical and can be sized so as to pass through a 15 mm trocar, a 12 mm trocar, or an 11 mm trocar. In the exemplary embodiment shown in FIGS. 1 and 2, the absorbent pad 130 generally comprises several layers of gauze sheets that have been rolled into a cylinder and secured, for example, with retaining member 132 (e.g., thread or suture). It will be appreciated that such gauze rolls can purchased pre-rolled (e.g., a Kittner roll marketed by Carefree Surgical Specialties, Inc.) or can be assembled, for example, by surgical staff. As shown in phantom in FIGS. 1 and 2, the absorbent pad 130 can surround a distal portion of the elongate shaft 110 such that the distal end 110b of the elongate shaft 110 partially extends within the absorbent pad 130 and such that the distal end 110b of the elongate shaft 110 is disposed between the proximal end 130a and distal end 130b of the absorbent pad 130.

As shown in FIGS. 1 and 2, a device in accordance with the present teachings additionally includes an actuation mechanism configured to move the absorbent pad from a first configuration (e.g., as shown in FIG. 1) to a folded or bent deployed configuration (as shown in FIG. 2). With specific reference to the exemplary device 100, the actuation mechanism 150 generally comprises a pull lever 152 that extends from the proximal end 110a of the elongate shaft 110, through an inner lumen thereof, out of the opening 112, and can be coupled to the distal end 130b of the absorbent pad 130. In some embodiments, the actuation mechanism 150 can additionally comprise a pull lever handle 154 to ease grasping thereof during deployment. It will also be appreciated that though the actuation of the actuating mechanism is generally described herein with reference to a manual force (e.g., by pulling), actuation can alternatively be accomplished through motor-driven means. Moreover, actuation can be accomplished by any other actuating mechanism known in the art and modified in accordance with the present teachings (e.g., via a scissor-type trigger handle, via rotation, etc.).

The pull lever 152 can have a variety of configurations and can be formed from a variety of materials (e.g., metal, plastic, nylon, thread) but generally is sufficiently strong such that a proximally directed force (e.g., on the pull lever handle 154) can be effective to manipulate the absorbent pad 130 as otherwise discussed herein. Moreover, as will be appreciated by a person skill in the art in light of the present teachings, the exemplary pull lever 152 exhibits relatively little stretch such that tension can be maintained on the absorbent pad 130 during deployment. Though the pull lever 152 can have a substantially uniform cross section along its entire length, in various aspects of the present teachings the pull lever 152 can have one or more features for locking the absorbent pad 130 in a deployed configuration. For example, as shown in FIGS. 1 and 2, for example, a portion of the pull lever 152 can be formed as a stop 156 having an increased diameter relative to the adjacent portions of the pull lever 152.

An exemplary deployment of the device 100 will now be described. In various aspects of the present teachings, a user of the device 100 can push the absorbent pad 130 through a trocar in the exemplary delivery configuration shown in FIG. 1. As shown, in this delivery configuration, the absorbent pad maintains a minimum cross-sectional area to ease insertion through the trocar. By way of example, the absorbent pad 130 is coupled to the distal end 110b of the shaft 110 such that the major dimension (length L) of the absorbent pad 130 is substantially aligned with the central axis of the elongate shaft 110, while the minor dimension (width W) is transverse to the longitudinal axis. Upon ejection of the absorbent pad 130 from the distal end of the trocar (e.g., through a distally directed force on the elongate shaft), the pull lever 152 can be actuated through a deployment force shown in FIG. 2. By pulling on the pull lever handle 154, the pull lever 152 will translate through the shaft 110, thereby pivoting or rotating the distal portion of the absorbent pad 130 in the direction of the arrow about the distal end 110a of the elongate shaft 110, thereby folding the absorbent pad 130 substantially on itself into the deployed configuration exemplified in FIG. 2. As indicated in FIG. 2, the absorbent pad has a reduced length (e.g., ½ L) in the deployed configuration and an increased width at the folded distal end 134. In various aspects, the pull force can be applied until the stop 156 enters into a compression fit engagement with the opening 112 of the elongate shaft 110, or alternatively, the entire stop 156 is pulled into the opening 112 with the distal shoulder 156b of the stop 156 abutting the inner surface of the opening 112. In this manner, tension on the absorbent pad can be maintained even after release of tension on the pull lever handle 154. Additionally or alternatively, it will be appreciated that one or more stops can be formed on a portion of the pull lever 152 (e.g., on the portion within the inner lumen) that can move proximally and be locked into a groove or cleat formed within the inner lumen.

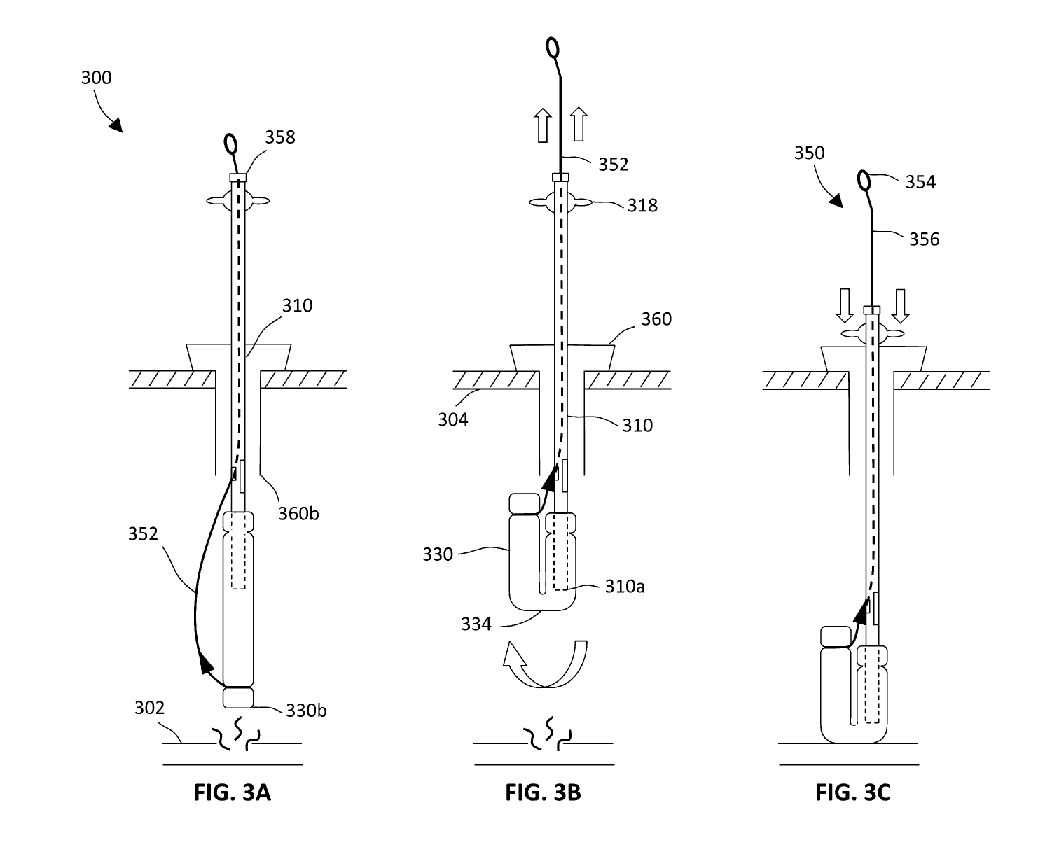

With reference now to FIGS. 3A-C, another exemplary device 300 in accordance with various aspects of the present teachings is schematically depicted being inserted through a trocar 360, which extends through a patient's skin surface. As shown in FIGS. 3A-C, the device 300 is substantially similar to that discussed above with reference to FIGS. 1 and 2, and generally includes an elongate shaft 310, an absorbent pad 330 disposed at a distal end thereof, and an actuation mechanism 350 for moving the absorbent pad 330 from the delivery configuration (FIG. 3A) to the deployment configuration (FIG. 3B). The device 300 differs, however, in that the actuation lever 352 includes a series of teeth 356 that are configured to engage with a locking mechanism 358, e.g., as with a ratchet and pawl. In this manner, upon proximal actuation of the pull lever 352, the engagement of the teeth 356 with a corresponding engagement feature of the locking mechanism 358 can be effective to maintain tension on the pull lever 352, thereby keeping the absorbent pad 330 in the deployed configuration. Though not shown, it will be appreciated by a person skilled in the art that the locking mechanism 358 can additionally include a release mechanism that can release the engagement with the teeth 356, thereby releasing tension on the absorbent pad 330 (e.g., for withdrawal).

As shown in FIGS. 3A-C, the elongate shaft 310 additionally includes elongated shaft handles 318 which can aid in the delivery of the absorbent pad 330 through the trocar 360, for example, until the absorbent pad is ejected out the distal end 360b thereof. For example, following the discovery of a hemorrhage in a blood vessel 302 that arose during a closed surgical procedure (e.g. laparoscopic or robot-assisted minimally invasive surgery), a user (e.g., surgeon, surgeon's assistant) can insert the device 300 into the proximal end of the trocar 360 (e.g., which can already be in place to aid in viewing the surgical site or can be inserted through the skin surface 304 upon discovery of the hemorrhage). The user can press, for example, on the elongated shaft handles 318 to move the distal end 310a of the elongate shaft distally, thereby pushing the absorbent pad 330 out of the distal end 360b of the trocar 360. Upon ejection of the absorbent pad 330, the pull lever 352 can be actuated through an upward (proximal) deployment force as shown in FIG. 3B, iteratively engaging the teeth. As above, by pulling on the pull handle 354, the pull lever 352 will translate through the shaft 310, thereby pivoting or rotating the distal portion of the absorbent pad 330 in the direction of the arrow about the distal end 310a of the elongate shaft 310 and folding the absorbent pad 330 substantially on itself into the deployed configuration exemplified in FIG. 3B. After manipulating the absorbent pad into the deployed configuration, an additional distally directed force on the elongated shaft handles 318 can maneuver the folded absorbent pad 330 (now with increased surface area) into contact with the vessel 302 at the hemorrhage site to slow or prevent blood loss therefrom. As shown, the absorbent pad has a reduced length along the central axis of the elongate shaft in the deployed configuration and an increased width at the folded distal end 334, thereby providing increased surface area (and additional absorptive material) relative to the distal end 330b in the delivery configuration. In various aspects, with the hemorrhage thus occluded, the surgeon can thus begin the conversion process, for example, by removing the robotic arms and making a conversion incision. It will be appreciated that though robotic arms may be used to slow the bleeding, the use of the exemplary devices and methods described herein can free both hands of the surgeon, for example, by having an assistant apply the tamponade.

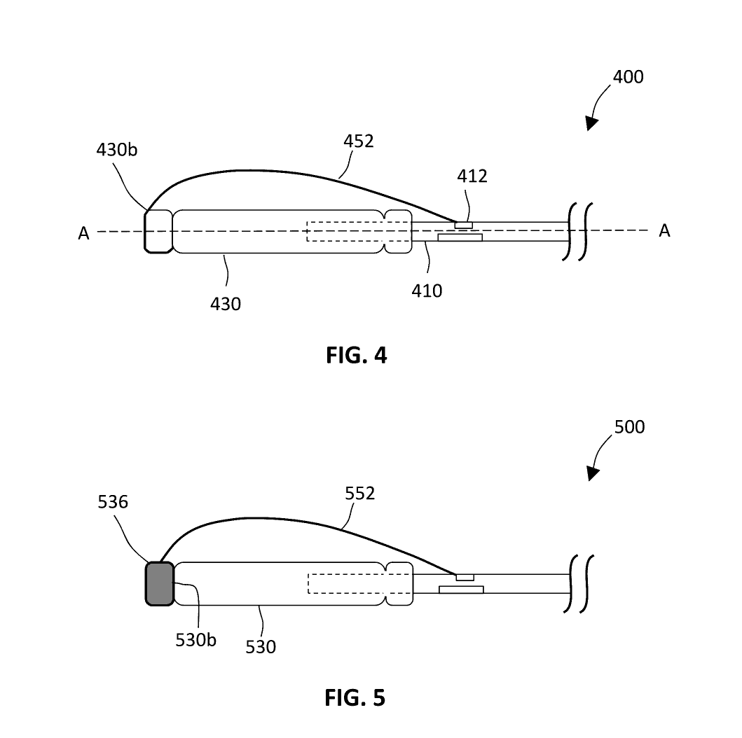

With reference now to FIG. 4, another exemplary device 400 in accordance with various aspects of the present teachings is depicted. FIG. 4 is substantially similar to the device 100 shown in FIGS. 1 and 2, but differs in that the pull lever 452 instead extends over the distal most end 430b of the absorbent pad 430 to couple to a portion of the absorbent pad 430 on an opposed side of the central axis (A) from the opening 412 in the elongate shaft 410.

With reference now to FIG. 5, another exemplary device 500 in accordance with various aspects of the present teachings is depicted. FIG. 5 is substantially similar to the device 100 shown in FIGS. 1 and 2, but differs in that the distal end or end area 530b of the absorbent pad 530 further comprises a distal cap 536 to which the pull lever 552 is coupled. It will be appreciated that the distal cap 536 can be formed from a different material from the remainder of the absorbent pad, for example, to provide a more rigid connection point for the pull lever 552 and/or to ease insertion of the absorbent pad 530 through a trocar.

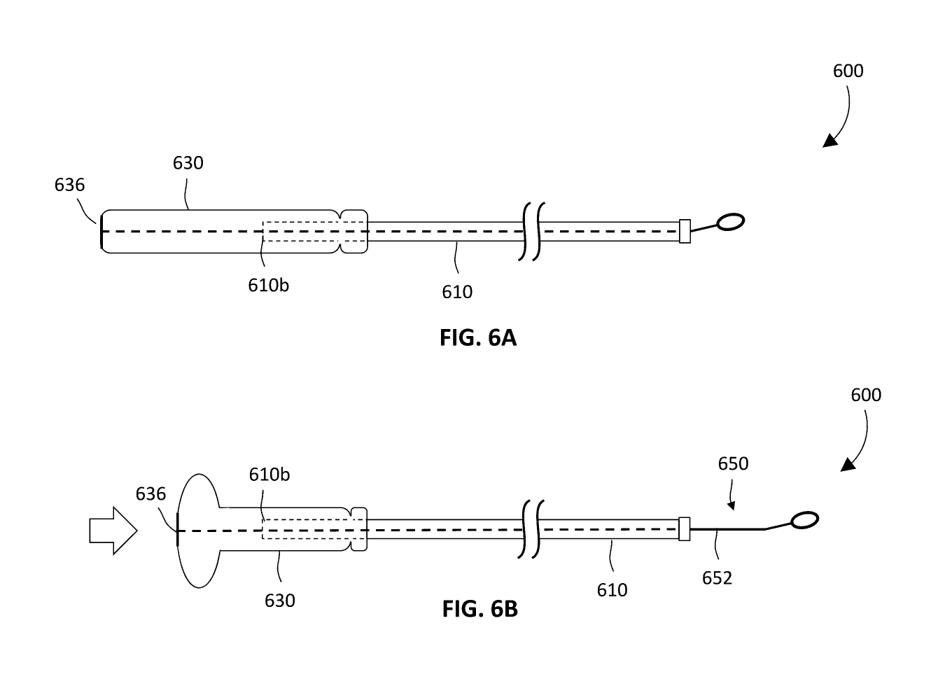

With reference now to FIGS. 6A and 6B, another exemplary device 600 in accordance with various aspects of the present teachings is depicted. The device 600 is similar to the device 500 discussed above with reference to FIG. 5 in that the device 600 also includes an elongate shaft 610, an absorbent pad 630 disposed at a distal end thereof and terminating distally in a distal end cap 636, and an actuation mechanism 650 for manipulating the absorbent pad 630. The device 600 differs, however, from the exemplary devices discussed above in that the pull lever 652 extends along the central axis of the elongate shaft 610 and out of the distal end 610b of the elongate shaft 610, and through the absorbent pad 630, terminating in a coupling with the distal end cap 636. In other words, unlike in the exemplary devices discussed above with reference to FIGS. 1-5, there is no proximal opening in the sidewall of the elongate shaft 610 through which the actuation lever 652 extends). Rather, by applying a proximally directed force via the pull lever 652 on the end cap 636 along the central axis of the elongate shaft 610, the end cap 636 is moved proximally thereby compressing the absorbent member 630 axially. In this manner, the absorbent member 630 can bunch about the distal end 610b of the elongate shaft, thereby radially expanding to provide an increased distal surface area for tamponade of the hemorrhaged vessel.

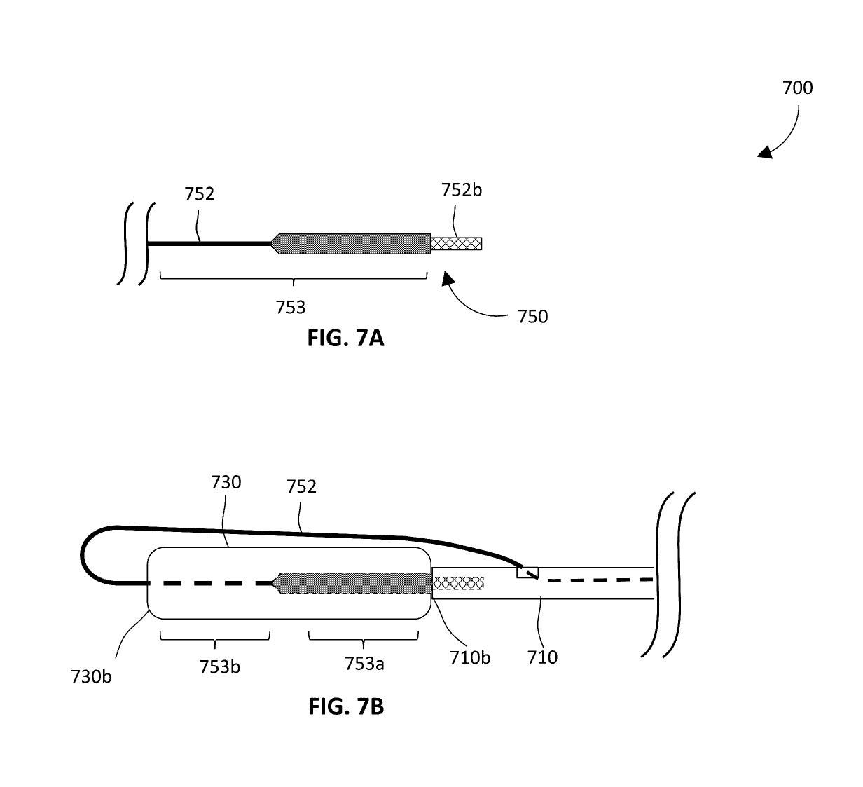

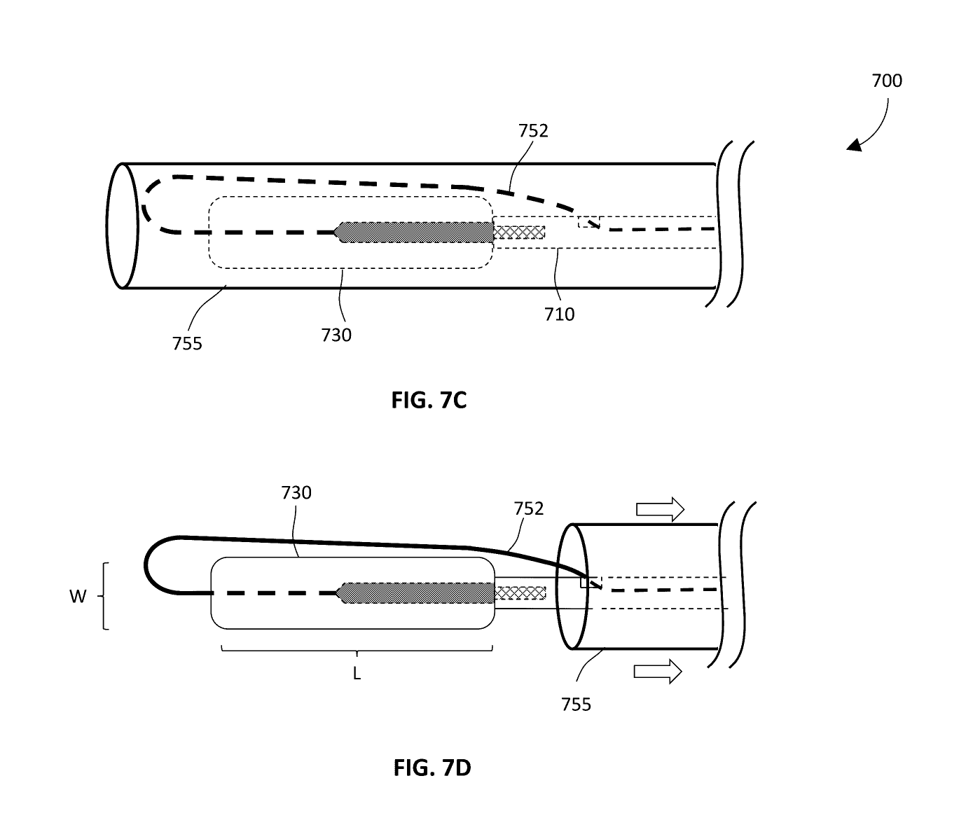

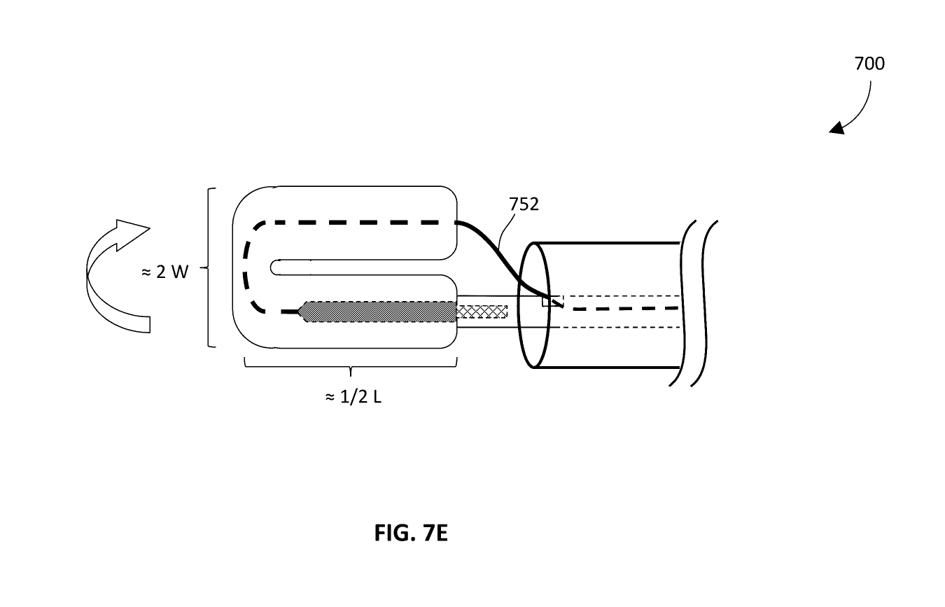

As noted above, actuation mechanisms in accordance with the present teachings can have a variety of configurations for moving an absorbent pad between a delivery configuration and a deployment configuration (e.g., folding the absorbent pad after positioning the absorbent pad within the surgical cavity). With reference now to FIGS. 7A and 7B, another exemplary device 700 in accordance with various aspects of the present teachings is schematically depicted. Like the devices discussed above, the device 700 includes an elongate shaft 710, an absorbent pad 730 disposed at a distal end thereof, and an actuation mechanism 750 for manipulating the absorbent pad 730. The device 700 differs, however, in that an actuation lever 752 of the actuation mechanism 750 extends from the distal end 710b of the elongate shaft 710 and through the absorbent pad 730, emerging therefrom at the distal end 730b of the pad. As best shown in FIGS. 7A and 7B, the pull lever 752 includes a terminal end 752b that can be secured to the hollow elongate shaft 710 adjacent its distal end 710b or a more proximal location. It will likewise be appreciated that the elongate pull lever 752 can be integrally formed with the elongate shaft 710 so as to extend from the distal end of the shaft through the absorbent pad 730 in accordance with various aspects of the present teachings. In use, the pull lever 752 can be pulled to cause bending of a distal portion of the pull lever and thus the surrounding absorbent pad 730 so as to provide a deployed configuration of the absorbent pad for the tamponade of a hemorrhage.

As noted above, in some embodiments, pull lever used in device according to the present teachings, can have a substantially uniform cross section along its entire length, and similarly can exhibit substantially constant rigidity along its length. In some embodiments, the thickness and/or rigidity of the pull lever can vary, e.g., in a continuous or discontinuous manner, along its length. By what of example, with specific reference to FIGS. 7A and 7B, the end of the pull lever 752 coupled to and/or extending from the distal end 710b of the elongate shaft can have various thicknesses and/or rigidities in order to facilitate the bending or folding of the absorbent pad 730 in accordance with various aspects of the present teachings. By way of example, a distal portion 753 of the pull lever 752 around which an absorbent pad 730 can be wrapped, secured, or disposed can have a segment 753a made of a stiffer material and/or can have an increased thickness relative to the material and/or thickness of the segment 753b. For example, the thickness of segment 753a can be at least 10% greater than segment 753b. It will be appreciated in light of the present teachings that actuation of the pull lever 752 will thus be more effective to bend the segment 753b relative to the segment 753a such that the folded absorbent pad 730 obtains its desired shape. In some aspects, by having the pull lever 752 extend through the entire length of the absorbent pad 730, the pull lever 752 can add to the rigidity and aid the delivery of the absorbent pad 730 through a trocar in the delivery configuration.

With specific reference now to FIGS. 7C-E, a thin sheath 755 can further be provided around the absorbent pad 730 and elongated shaft 710 to ease the insertion and/or deployment of the device. The thin sheath 755 can be used to cover absorbent pad 730 and at least a portion of the pull lever 752 and the elongated shaft 710 when the device is in the loaded configuration as illustrated in FIG. 7C. In this configuration, the thin sheath 755 can reduce frictional forces as the absorbent pad 730 and the elongated shaft 710 are inserted through the trocar. In some embodiments, the thin sheath 755 can be used to cover partially or completely the elongated shaft 710 when the device is in the delivery and deployment configurations as illustrated in FIGS. 7D and 7E, respectively, while leaving the thin sheath 755 proximally withdrawn from the thin sheath 755. In these configurations, the thin sheath 755 can reduce the frictional forces as the absorbent pad 730 is compressed radially as it passes through the trocar. Furthermore, the absorbent pad 730 can be actuated into its deployed configuration by bending it so that its proximal and distal ends meet. As a result, in the deployed configuration the absorbent pad can have a reduced length (e.g., ½ L) and an increased width (e.g., 2 W), providing a larger surface area to cover the hemorrhage.

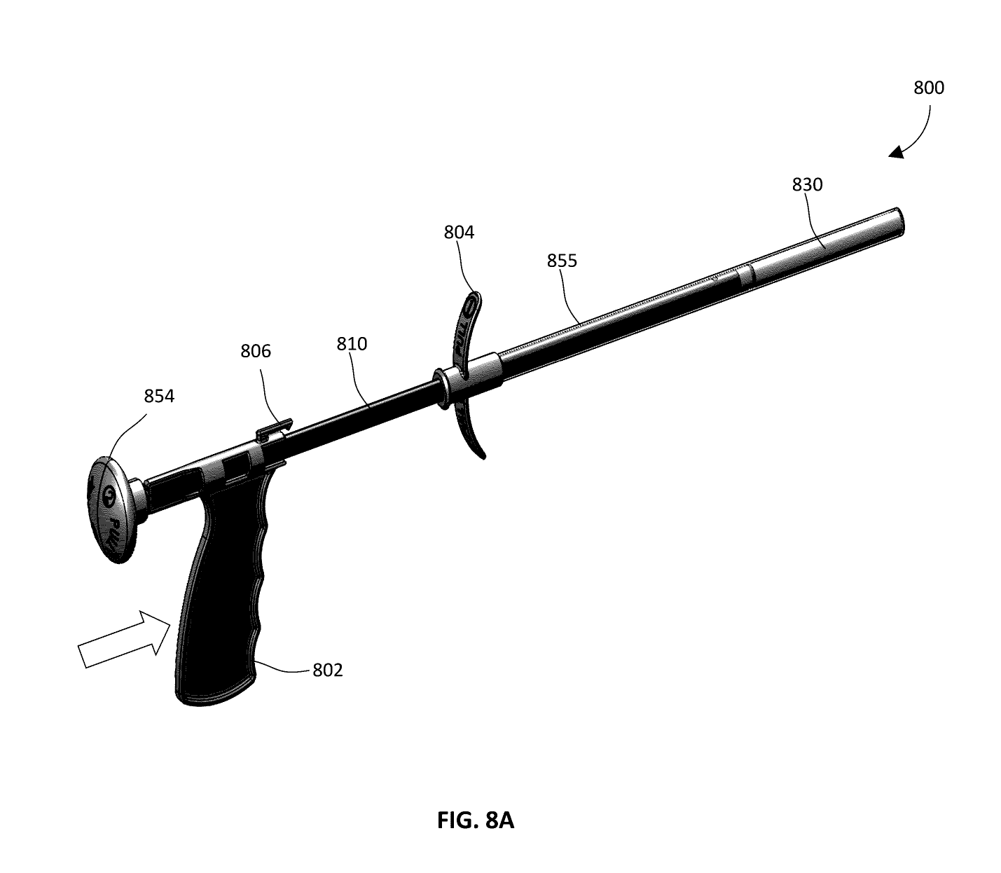

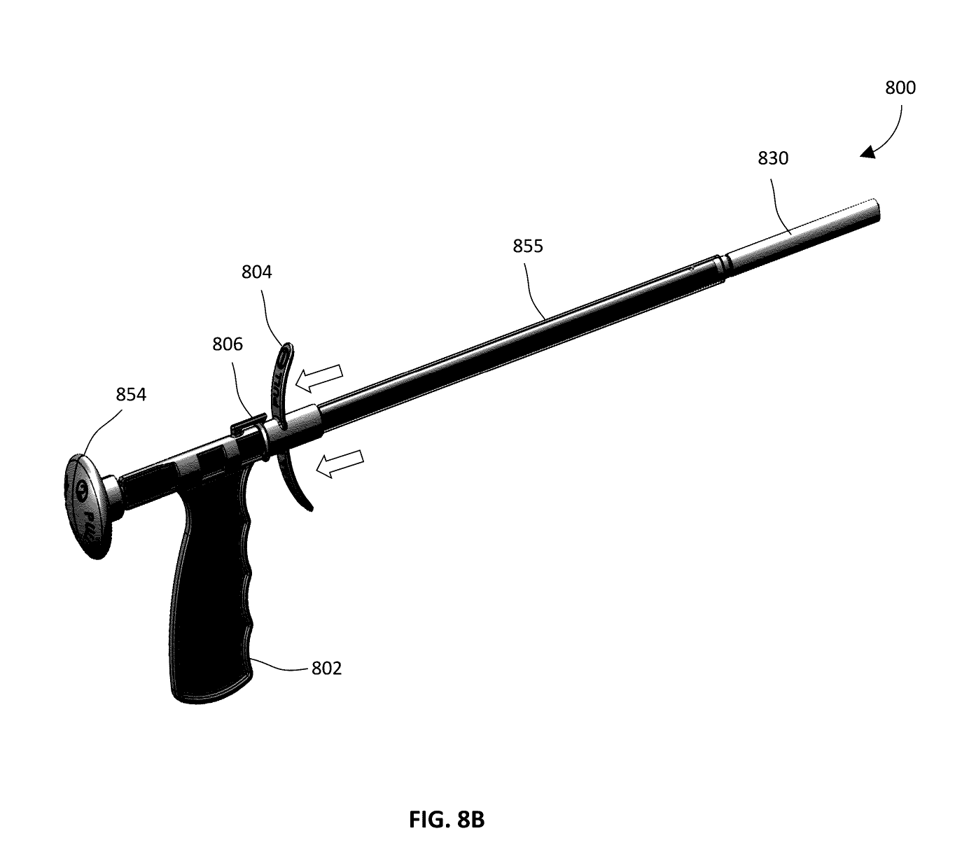

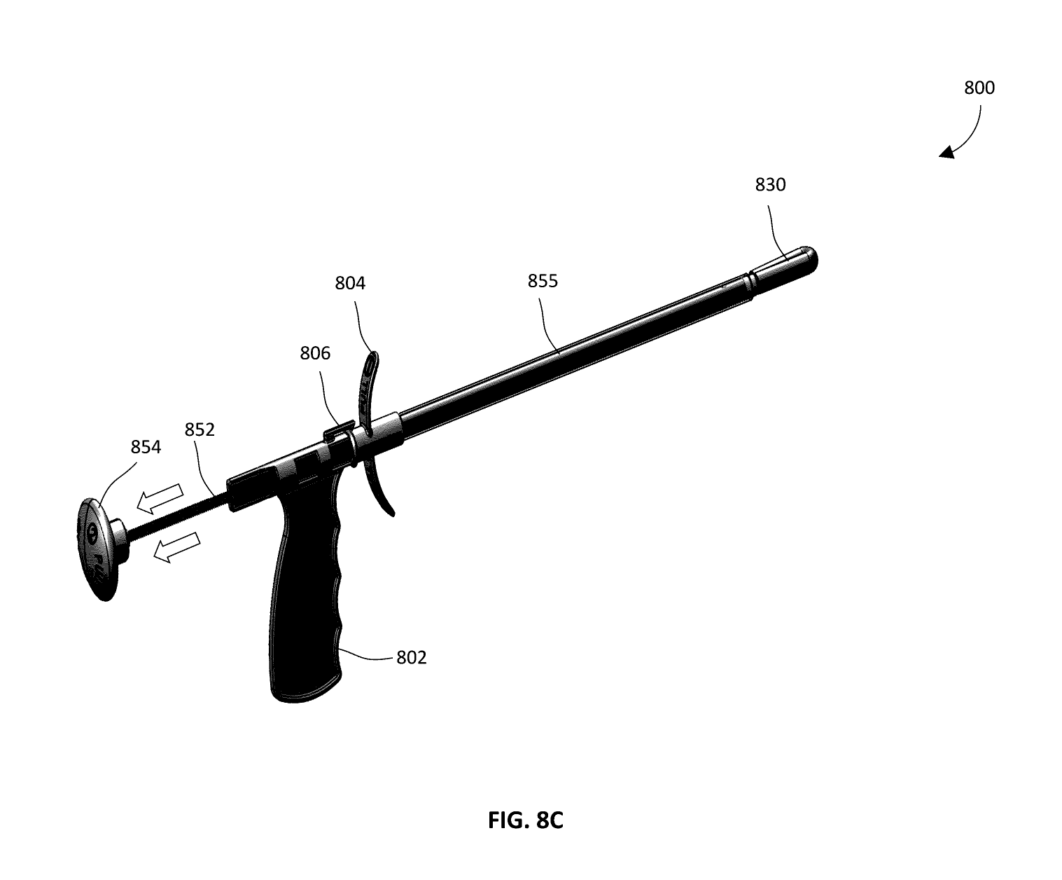

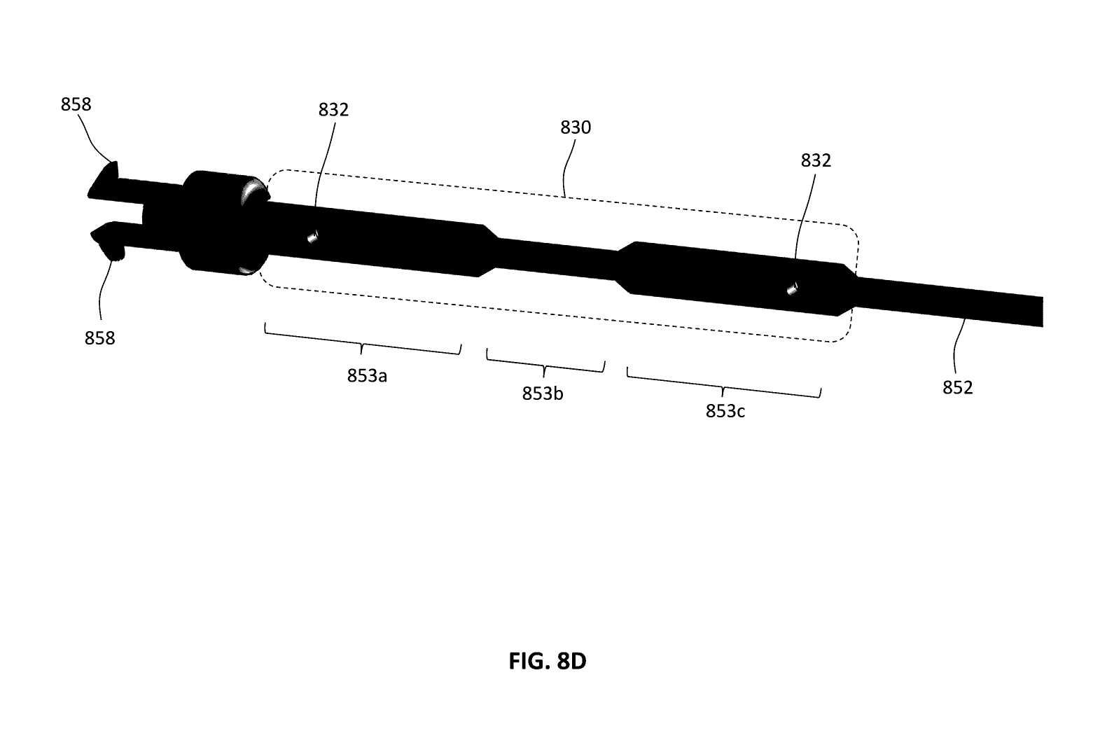

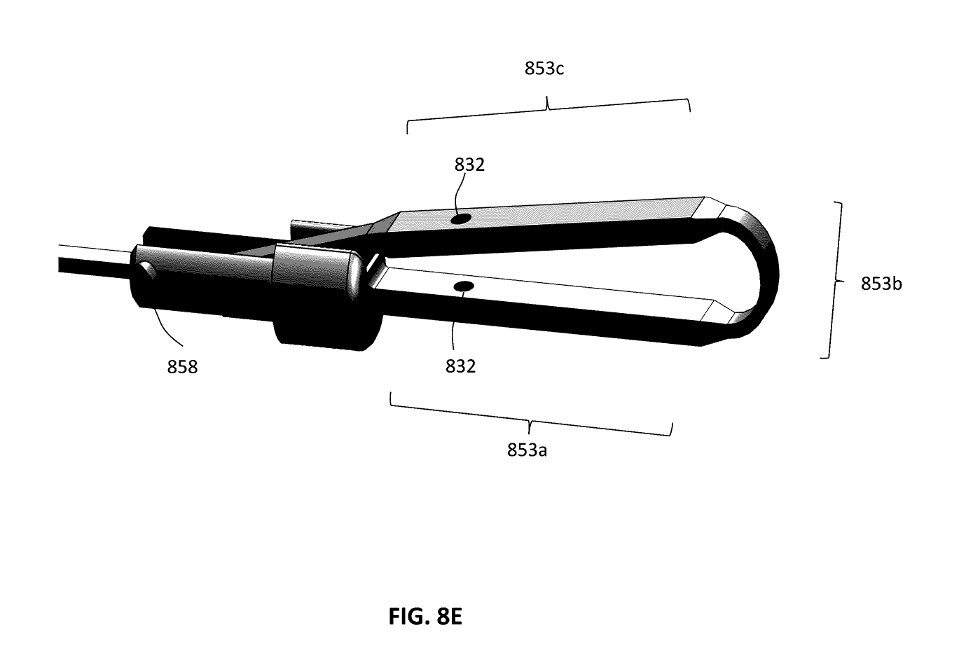

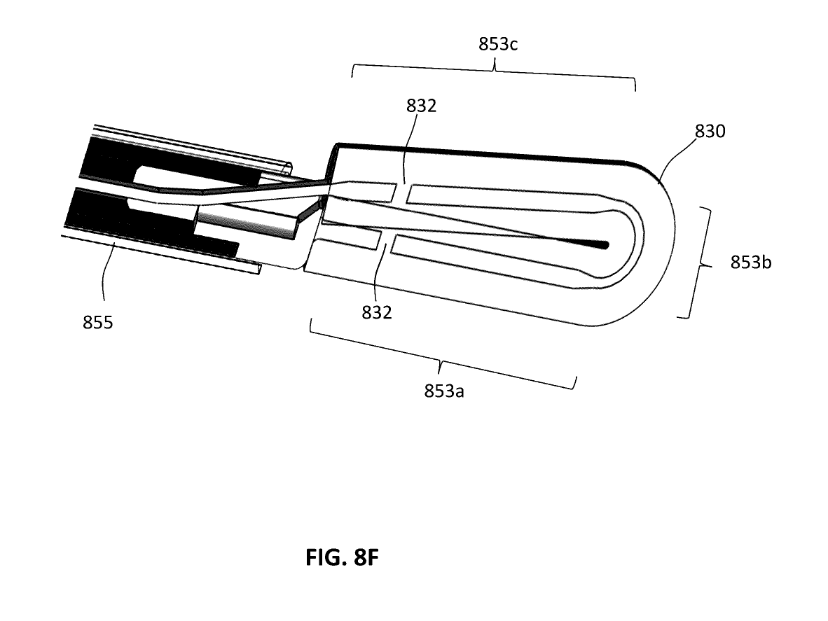

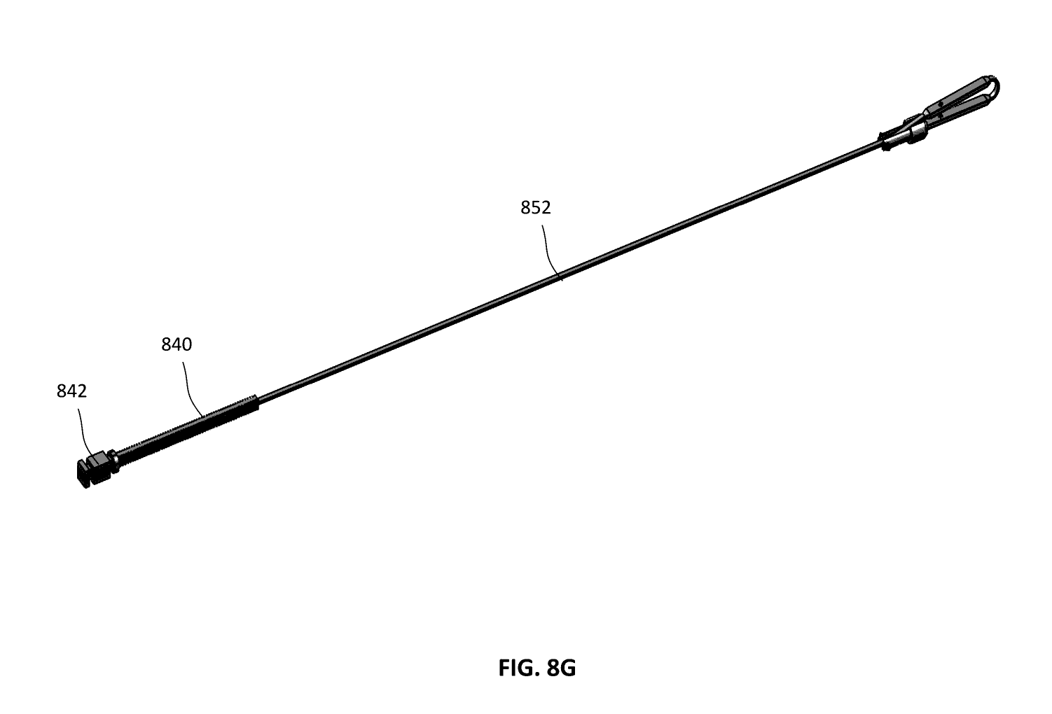

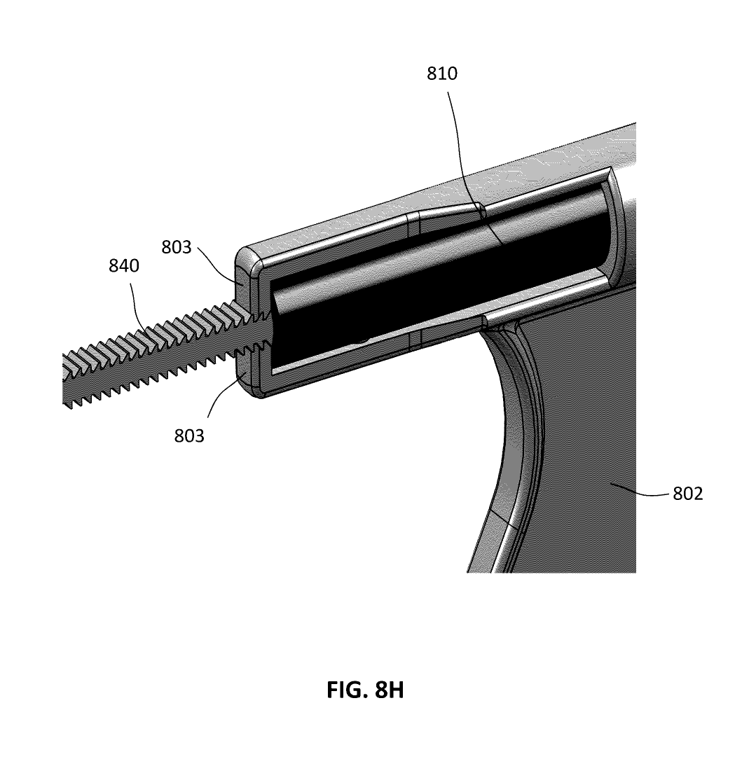

With reference now to FIGS. 8A-H, another exemplary device 800 for applying tamponade to an internal hemorrhage site in accordance with various aspects of the present teachings is schematically depicted. As shown, the device 800 includes a pistol grip 802, an elongate shaft 810, an absorbent pad 830 disposed at a distal end of the shaft 810, a pull lever 852 extending through the shaft 810, and a pull lever handle 854 for moving the absorbent pad 830 between a delivery configuration and a deployed configuration, as discussed otherwise herein. As shown in FIG. 8A and 8B, the device 800 additionally includes a sheath 855 and a sheath handle 804 coupled to the proximal end thereof, the sheath 855 configured to be disposed to surround the elongate shaft 810 and the absorbent pad 830 during insertion through a trocar (as in FIG. 8A). As shown in FIG. 8B, the sheath handle 804 can then be actuated (e.g., by pulling the sheath handle proximally) to withdraw the sheath from around the absorbent pad 830 when a user is ready to deploy the absorbent pad 830. As best shown in FIG. 8B, the sheath handle 804 can be engaged by locking mechanism 806 so as to maintain the sheath 855 in the withdrawn position.

Once the thin sheath handles 810 are locked, an actuation mechanism can then be actuated so as to move the absorbent pad 830 from its delivery configuration (extending along longitudinal axis of the elongate shaft 810 (as in FIG. 8B) to its deployed configuration (as in FIG. 8C). As shown in FIG. 8C, for example, pull lever handle 854 can be pulled proximally to fold the absorbent pad 830. In some embodiments, the pull lever 852 can have a substantially uniform cross section along its length. In other embodiments, the pull lever 852 can have regions exhibiting different thickness or rigidity. It will be appreciated in light of the present teachings that the pull lever 852 can be formed of a single material or can include different regions formed of materials exhibiting different rigidities. By way of example and with specific reference to FIGS. 8D-8F, the distal portion of the pull lever 852 (which extends through the absorbent pad 830 as shown in phantom) includes two sections 853a and 853c that are thicker and/or more rigid than section 853b disposed therebetween, in order to facilitate the folding of the absorbent pad 830. In this manner, the section 853b can function as a hinge such that a proximally-directed force on the pull lever 852 selectively bends the pull lever 852 at section 853b, thereby folding the absorbent pad 830 disposed therearound, as illustrated in FIGS. 8E and 8F. In various aspects, the absorbent pad can have a length in the range of about 4 to about 10 centimeters, with the hinge portion 830 having a length in a range of about 1 to about 4 centimeters, by way of non-limiting example.

Referring again to FIGS. 8D-8F, the distal portion of the pull lever 852 comprises one or more throughholes 832 that can be utilized to couple the absorbent pad thereto (e.g., by sewing the pad to the pull lever 852). For example, as shown in FIG. 8D, after wrapping or disposing the absorbent pad 830 about the distal portion of the pull lever, the absorbent pad 830 can be firmly coupled to the pull lever by threading a suture through the absorbent pad and the throughholes 832 in sections 853a and 853b. Referring still to FIG. 8D, the distal-most end of the pull lever 852 can also comprise a coupling mechanism 858 to couple the distal-most end of the pull lever 852 to the distal end of the elongated shaft 810. By way of example, an internal surface of the elongated shaft 810 can comprise recesses configured to receive the coupling mechanism 858 (e.g., pins). The coupling between the elongated shaft 810 and the coupling mechanism 858 of the distal end of the pull lever 852 is illustrated in the FIG. 8F, which depicts a cross section of the distal end of the device 800 in the deployed configuration. It will be appreciated that in its delivery configuration, the pull lever 852 would therefore extend through the distal end of the elongate shaft 810, along an external surface of the absorbent pad 830 (e.g., between the absorbent pad 830 and the sheath 855), into the distal end of the absorbent pad 830, through the absorbent pad 830, out of the proximal end of the absorbent pad 830, and is coupled to the distal end of the elongate shaft 810 via coupling mechanism 858. As with the hinge section 853b, the connection between section 853c and the thinner/less rigid section of the pull lever 852 is configured to allow the pull lever 852 to bend thereat such that the pull lever 852 emerges from the distal end of the absorbent pad 830 in the delivery configuration and reverses direction to extend proximally through the elongate shaft 810.

With reference now referring to FIGS. 8G and 8H, the proximal end of the pull lever 852 is shown in additional detail. As shown, the proximal portion of the pull lever 852 can comprise a plurality of teeth 840 and proximally terminate in a pull lever handle hook 842 that can be coupled to pull lever handle 854 for actuating the pull lever 852. As best shown in FIG. 8H, the plurality of teeth 840 can be configured to couple with a ratchet mechanism 803 of the handle 802 so as to maintain the pull lever 852 in place when the absorbent pad 830 is in its deployment configuration. It will be appreciated that the ratchet mechanism 803 can include release mechanism (not shown) to release the pull lever 852, for example, to straighten the absorbent pad for extraction.

Those skilled in the art will know or be able to ascertain using no more than routine experimentation, many equivalents to the embodiments and practices described herein. Accordingly, it will be understood that the invention is not to be limited to the embodiments disclosed herein, but is to be understood from the following claims, which are to be interpreted as broadly as allowed under the law.

The section headings used herein are for organizational purposes only and are not to be construed as limiting. While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

What is claimed:

1. A device for applying tamponade to an internal hemorrhage site during a laparoscopic procedure, comprising:
an elongate shaft extending along a longitudinal axis from a proximal end to a distal end;
an absorbent pad disposed at the distal end of the elongate shaft, the absorbent pad extending in a delivery configuration along the longitudinal axis from a proximal end to a distal end; and
an actuation mechanism extending from the proximal end of the elongate shaft to the absorbent pad, wherein actuation of the actuation mechanism is configured to move the absorbent pad from the delivery configuration to a deployed configuration for applying tamponade to a hemorrhage site.

2. The device of claim 1, wherein the actuation mechanism is configured to fold the absorbent pad.

3. The device of claim 2, wherein, in the deployed configuration, the absorbent pad is folded such that the distal and proximal ends of the absorbent pad are disposed adjacent one another.

4. The device of claim 1, wherein the absorbent pad exhibits a first length along the central longitudinal axis in the delivery configuration and a second length along the central longitudinal axis in the deployed configuration, the first length being greater than the second length.

5. The device of claim 1, wherein a distal portion of the absorbent pad has an increased thickness in the deployed configuration relative to the proximal end of the absorbent pad in the delivery configuration.

6. The device of claim 1, wherein a proximal portion of the absorbent pad is disposed about the distal end of the elongate shaft and wherein the actuation mechanism is configured to rotate a distal portion of the absorbent pad about the distal end of the elongate shaft. The device of claim 1, wherein a distal portion of the actuation mechanism extends through the absorbent pad and is coupled to the distal end of the elongate shaft.

8. The device of claim 7, wherein the distal portion of the actuation mechanism is sufficiently rigid to aid delivery of the absorbent pad through a trocar extending through a patient's skin surface.

9. The device of claim 7, wherein the distal portion of the actuation mechanism extending through the absorbent pad comprises at least two segments having different rigidities.

10. The device of claim 9, wherein the at least two segments having different rigidities function as a hinge within the absorbent pad upon actuation of the actuation mechanism.

11. The device of claim 1, wherein the actuation mechanism extends through the distal end of the elongate shaft, along an external surface of the absorbent pad, into the distal end of the absorbent pad, through the absorbent pad, out of the proximal end of the absorbent pad, and is coupled to the distal end of the elongate shaft.

12. The device of claim 1, wherein the actuation mechanism extends through the absorbent pad and is coupled to a distal end thereof, and wherein the actuation mechanism is configured to pull the distal end of the absorbent pad toward the proximal end of the absorbent pad substantially along the longitudinal axis.

13. The device of claim 1, wherein the absorbent pad comprises gauze rolled to form a cylinder.

14. The device of claim 1, wherein the absorbent pad comprises sponge.

15. The device of claim 1, further comprising a lumen extending through the elongate shaft and in fluid communication with the absorbent pad, wherein the lumen is configured to couple to a negative pressure source for application of suction through the absorbent pad.

16. The device of claim 1, further comprising a lumen extending through the elongate shaft and in fluid communication with the absorbent pad, wherein the lumen is configured to couple to a fluid source for application of hemostatic agents to the hemorrhage site through the absorbent pad.

17. A device for applying tamponade to an internal hemorrhage site during a laparoscopic procedure, comprising:
an elongate shaft extending along a longitudinal axis from a proximal end to a distal end;
an absorbent pad coupled to the distal end of the elongate shaft, the absorbent pad extending in a delivery configuration along the longitudinal axis from a proximal end to a distal end; and
an actuation mechanism extending from the proximal end of the elongate shaft to the absorbent pad, wherein actuation of the actuation mechanism is configured to move the distal end of the absorbent pad toward the proximal end of the elongate shaft.

18. A method of applying tamponade to an internal hemorrhage site during a laparoscopic procedure, comprising:
delivering an absorbent pad in a first configuration through a trocar extending through a skin surface to an internal hemorrhage site, the absorbent pad being disposed at a distal end of an elongate shaft extending along a longitudinal axis from a proximal end to the distal end, wherein the absorbent pad extends substantially along the longitudinal axis from a proximal end to a distal end in the first configuration;
deploying the absorbent pad by moving the absorbent pad from the first configuration to a second configuration; and
applying pressure to the internal hemorrhage site with the absorbent pad in the second configuration.

19. The method of claim 18, wherein deploying the absorbent pad comprises folding the absorbent pad such that the distal and proximal ends of the absorbent pad are disposed adjacent one another.

20. The method of claim 18, wherein deploying the absorbent pad comprises actuating a pull lever coupled to the distal end of the elongate shaft so as to bend a portion of the pull lever extending through the absorbent pad.

21. The method of claim 18, further comprising converting the laparoscopic procedure to an open procedure while maintaining pressure to the internal hemorrhage site with the absorbent pad in the second configuration.

22. The method of claim 21, wherein the laparoscopic procedure comprises a robot-assisted laparoscopic procedure, wherein the method further comprises disengaging the robot from the patient prior to making a conversion incision.
